(19) United States
(12) Patent Application Publication
(10) Pub. No.: US 2019/0391309 A1
(43) Pub. Date: Dec. 26, 2019

(54) PHOTOCHROMIC ANTI-FOG LENS AND METHOD FOR MAKING THE SAME

(71) Applicant: KODA OPTICAL CO., LTD., TAINAN (TW)

(72) Inventor: Chih Ming WU, TAINAN (TW)

(73) Assignee: KODA OPTICAL CO., LTD., TAINAN (TW)

(21) Appl. No.: 16/016,674

(22) Filed: Jun. 25, 2018

(51) Int. Cl. G02B 5/23; G02B 1/18; B29D 11/00; A61F 9/02

(52) U.S. Cl. CPC G02B 5/23; G02B 1/18; G02C 7/102; A61F 9/029; B29D 11/00009

(57) ABSTRACT

A photochromic anti-fog lens and a method for making a photochromic anti-fog lens includes a step of mixing photochromic material in glue which is used to glue the second transparent plastic plate to the inner side of the first transparent plastic plate to get a photochromic material layer between the first and second transparent plastic plates. A step of attaching an anti-fog film to the inner side of the second transparent plastic plate to form a photochromic anti-fog composite lens. A step of putting the photochromic anti-fog composite lens in the cavity of a molding set. A step of injecting melted lens-forming material into the cavity of the molding set, the melted lens-forming material connected with the first transparent plastic plate to form the photochromic anti-fog lens that includes multiple layers with high structural strength.

### BACKGROUND OF THE INVENTION

### 1. Fields of the Invention

The present invention relates to a photochromic anti-fog lens and the method for making the photochromic anti-fog lens.

### 2. Descriptions of Related Art

The conventional lenses used as helmet goggles or ski goggles generally are made by Polycarbonate. However, Polycarbonate does not have anti-fog feature which is important for goggles and the like.

In order to have the anti-fog feature, one of the conventional way is to attach an anti-fog film at the inner side of the lens, while the lens still has normal function that allows light to pass the lens. Another way is to spray a layer of photochromic material on the outer side of the lens, and the photochromic material changes its color when sensing light to protect the user's eyes. The lenses are call photochromic lenses.

Nevertheless, the lenses made of Polycarbonate has a smooth and hard inner side which is difficult to be attached with the TAC anti-fog film. Air can easily establish bubbles on the TAO anti-fog film to affect the features and appearances of the lenses. Besides, the TAC anti-fog film is exposed and attached on the outer side of the lenses can easily be damaged or peeled off. Therefore, a lens that includes the features of anti-fog and photochromic discoloration is needed.

The present invention is intended to provide a photochromic anti-fog lens and the method for making the photochromic anti-fog lens. The lenses of the present invention are stable and durable.

### SUMMARY OF THE INVENTION

The present invention relates to a photochromic anti-fog lens which comprises a lens and a photochromic anti-fog composite lens. The photochromic anti-fog composite lens includes a first transparent plastic plate and a second transparent plastic plate which is glued to the inner side of the first transparent plastic plate by glue. The glue includes photochromic material. The second transparent plastic plate has an anti-fog film attached to the inner side thereof. The lens is formed in a molding set and securely connected with the first transparent plastic plate located in a cavity of the molding set located beside the inner side of the first transparent plastic plate.

The present invention also provides a method for making a photochromic anti-fog lens, and the method comprising:

a step 1: mixing photochromic material in glue;

a step 2: spreading the glue onto the inner side of a first transparent plastic plate and/or the outer side of the second transparent plastic plate, attaching the second transparent plastic plate to the inner side of the first transparent plastic plate, attaching an anti-fog film on the inner side of the second transparent plastic plate to form a photochromic anti-fog composite lens;

a step 3: putting the photochromic anti-fog composite in a cavity of a molding set, the first transparent plastic plate located at the inner side of a lens to be formed, and

a step 4: injecting a melted lens-forming material into the cavity of the molding set, the melted lens-forming material connected with the first transparent plastic plate to form a photochromic an fog lens that includes the lens connected to the photochromic anti-fog composite lens.

The present invention provides a glue that has photochromic material mixed therein, and the second transparent plastic plate is connected to the inner side of the first transparent plastic plate by the glue such that a photochromic material layer is formed between the first and second transparent plastic plates. An anti-fog film is attached on the inner side of the second transparent plastic plate to form a photochromic anti-fog composite lens. The photochromic anti-fog composite is then put in a cavity of a molding set, and the first transparent plastic plate is located at the inner side of a lens to be formed in the molding set. A melted lens-forming material at a temperature of 260 to 280 degrees Celsius is then injected into the cavity of the molding set, and the melted lens-forming material is connected with the first transparent plastic plate to form a photochromic anti-fog lens. The photochromic anti-fog lens includes a structure of multiple layers. The first and second transparent plastic plates are formed as an integral plate and do not separate from each other. The photochromic material layer is formed between the first and second transparent plastic plates so as to avoid from being exposed to the air and foreign objects, and the photochromic material layer is permanently protected between the first and second transparent plastic plates without worry of peeling off and being damaged.

The present invention will become more obvious from the following description when taken in connection with the accompanying drawings which show, for purposes of illustration only, a preferred embodiment in accordance with the present invention.

### BRIEF DESCRIPTION OF THE DRAWINGS

FIG. 1 is a perspective view to show the photochromic anti-fog lens of the present invention;

FIG. 2 is an exploded view of the photochromic anti-fog lens of the present invention;

FIG. 3 is a perspective view to show the second embodiment of the photochromic anti-fog lens of the present invention, and

FIG. 4 is an exploded view to show the second embodiment of the photochromic anti-fog lens of the present invention.

### DETAILED DESCRIPTION OF THE PREFERRED EMBODIMENT

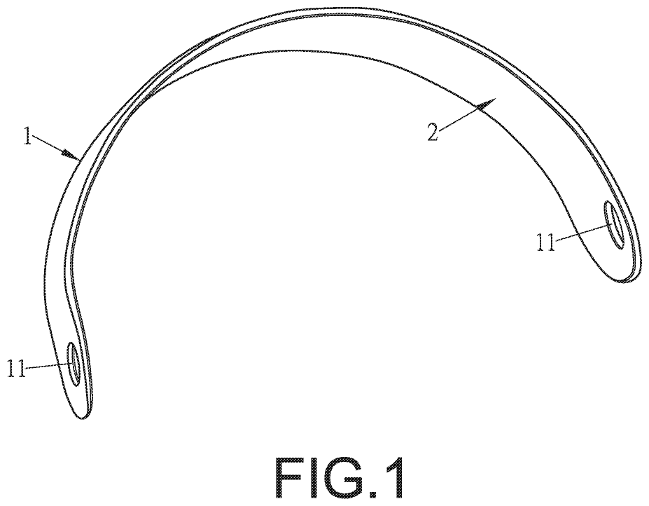

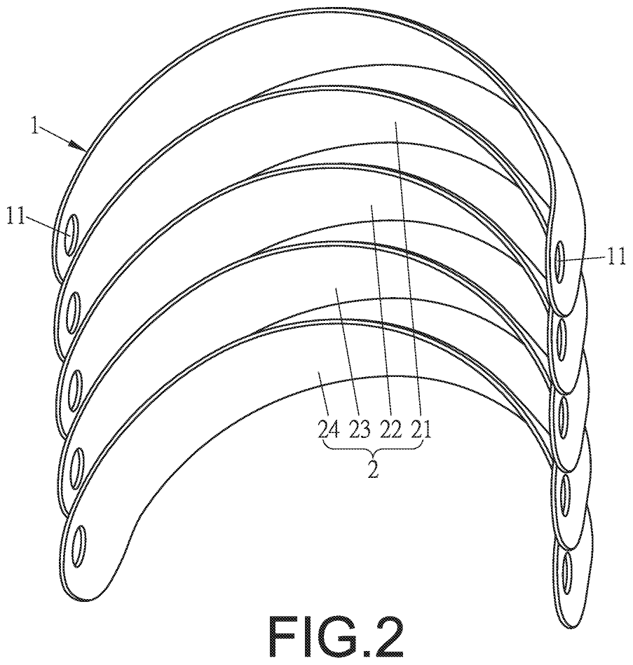

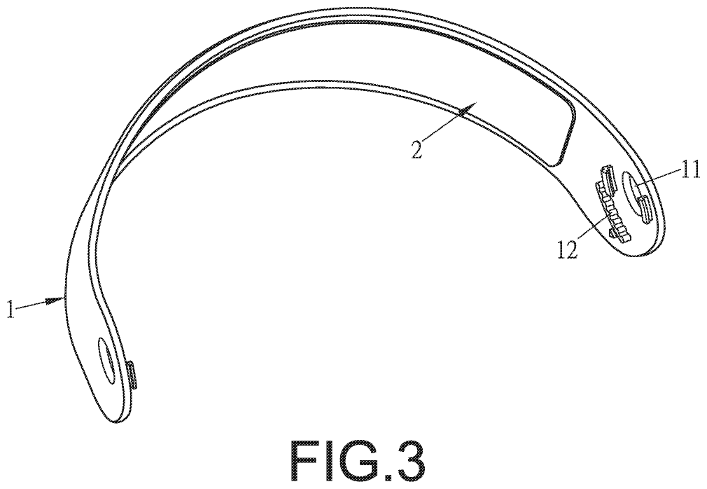

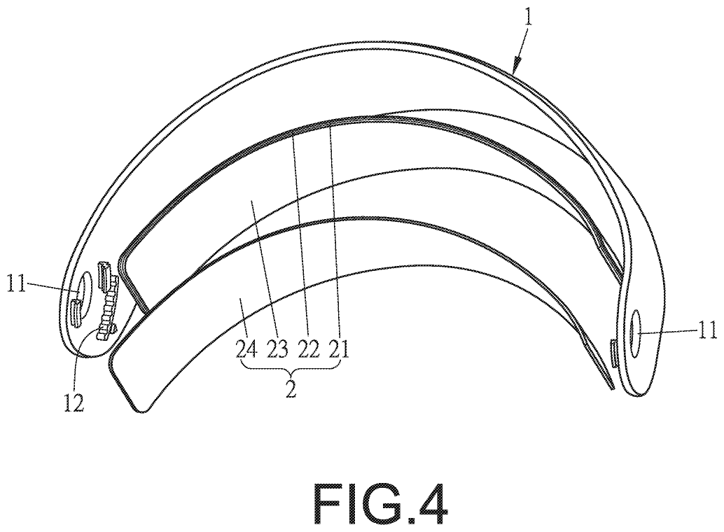

Referring to FIGS. 1 to 4, the photochromic anti-fog lens of the present invention comprises a lens 1 and a photochromic anti-fog composite lens 2. The lens 1 can be used as helmet goggles or ski goggles or normal and functional glasses.

The photochromic anti-fog composite lens 2 comprises a first transparent plastic plate 21 and a second transparent plastic plate 23. The second transparent plastic plate 23 is glued to the inner side of the first transparent plastic plate 21 by glue. The glue has photochromic material mixed therein. An anti-fog film 24 is attached to the inner side of the second transparent plastic plate 23. A photochromic material layer 22 is formed between the first and second transparent plastic plates 21, 23 after the glue is dried. The photochromic material should be mixed with the glue as much as possible so as to ensure the photochromic features. The lens 1 is formed in a molding set which is not shown, and securely connected with the first transparent plastic plate 21 located in a cavity of the molding set located beside the inner side of the first transparent plastic plate 21. Specifically, the melted lens-forming material which can be Polycarbonate or Polymer resin, and has a temperature of 260 to 280 degrees Celsius, is injected into the cavity of the molding set, and the melted lens-forming material is connected with the first transparent plastic plate to form a photochromic anti-fog lens. The first and second transparent plastic plates 21, 23 are formed as an integral plate and do not separate from each other. The photochromic material layer 22 is formed between the first and second transparent plastic plates 21, 23 to avoid from being exposed to the air and foreign objects, and the photochromic material layer 22 is permanently protected between the first and second transparent plastic plates 21, 23 without worry of peeling off and being damaged.

In the first embodiment of the present invention, the first and second transparent plastic plates 21, 23 are made of Polycarbonate so that the first and second transparent plastic plates 21, 23 are thin and can be firmly connected to each other. The first and second transparent plastic plates 21, 23 can also be made by other material such as Polypropylene. Specifically, the first transparent plastic plate 21 and the second transparent plastic plate 23 each have a thickness of 0.1 mm to 0.5 mm, such as 0.1 mm, 0.15 mm, 0.2 mm, 0.25 mm, 0.3 mm, 0.35 mm, 0.4 mm, 0.45 mm, or 0.5 mm.

The photochromic material and the anti-fog film 24 both are well-known materials in this field. The anti-fog film 24 can be made of Triacetate fiber film (TAC film). The TAC film can be well connected with a surface that has less smoothness and hardness of the second transparent plastic plate 23, such that the anti-fog film 24 does not separated from the second transparent plastic plate 23.

Furthermore, first and second transparent plastic plates 21, 23 have photochromic material mixed therein so as to perform better photochromic features for the photochromic anti-fog composite lens 2.

When the lens 1 and the photochromic anti-fog composite lens 2 each are a flat lens, the photochromic anti-fog composite lens 2 can be directly put in the cavity of the molding set to be combined with the lens 1. When the lens 1 is a curved lens and the photochromic anti-fog composite lens 2 is a flat lens, then the photochromic anti-fog composite lens 2 is processed to be curved before the photochromic anti-fog composite lens 2 is put into the cavity of the molding set such that the photochromic anti-fog composite lens 2 is connected to the lens 1.

When the lens 1 be used as helmet goggles or ski goggles, the lens 1 includes an installation hole 11 defined in each of two ends thereof so as to be connected to the helmet frame or the goggle frame (not shown). The photochromic anti-fog composite lens 2 is overlapped to the inner side of the lens 1, wherein the photochromic anti-fog composite lens 2 includes two holes (not shown) which are located corresponding to the two installation holes 11. Alternatively, when the photochromic anti-fog composite lens 2 is overlapped to an area between the two installation holes 11 of an inner side of the lens 1, then the photochromic anti-fog composite lens 2 does not have the holes. An adjustment device 12 protrudes from the inner side of the lens 1 and located beside each of the two installation holes 11 so as to adjust the angle of the photochromic anti-fog lens.

The present invention provides a method for making a photochromic anti-fog lens, and the method comprises:

a step 1: mixing photochromic material in glue;

The photochromic material is Ultraviolet photochromic material which is mixed with the glue as much as possible.

a step 2: spreading the glue onto the inner side of a first transparent plastic plate 21 and/or the outer side of the second transparent plastic plate 23, attaching the second transparent plastic plate 23 to the inner side of the first transparent plastic plate 21, attaching an anti-fog film 24 on the inner side of the second transparent plastic plate 23 to form a photochromic anti-fog composite lens 2;

The anti-fog film 24 is the Triacetate fiber film (TAO film), and the thickness of each of the first and second transparent plastic plates 21, 23 is 0.1 mm to 0.5 mm. A photochromic material layer 22 is formed between the first and second transparent plastic plates 21, 23 after the glue is dried. The anti-fog film 24 can be well connected with a surface that has less smoothness and hardness of the second transparent plastic plate 23, such that the anti-fog film 24 does not separated from the second transparent plastic plate 23.

a step 3: putting the photochromic anti-fog composite lens 2 in a cavity of a molding set, the first transparent plastic plate 21 located at an inner side of a lens 1 to be formed in the molding set;

When the lens 1 and the photochromic anti-fog composite lens 2 each are a flat lens, the photochromic anti-fog composite lens 2 can be directly put in the cavity of the molding set to be combined with the lens 1. When the lens 1 is a curved lens and the photochromic anti-fog composite lens 2 is a flat lens, then the photochromic anti-fog composite lens 2 is processed to be curved before the photochromic anti-fog composite lens 2 is put into the cavity of the molding set such that the photochromic anti-fog composite lens 2 is connected to the lens 1.

a step 4: injecting a melted lens-forming material into the cavity of the molding set, the melted lens-forming material connected with the first transparent plastic plate 21 to form a photochromic anti-fog lens that includes the lens 1 connected to the photochromic anti-fog composite lens 2;

When the lens 1 be used as helmet goggles or ski goggles, the lens 1 includes an installation hole 11 defined in each of two ends thereof so as to be connected to the helmet frame or the goggle frame (not shown). The photochromic anti-fog composite lens 2 is overlapped to the inner side of the lens 1, wherein the photochromic anti-fog composite lens 2 includes two holes (not shown) which are located corresponding to the two installation holes 11. Alternatively, when the photochromic anti-fog composite lens 2 is overlapped to an area between the two installation holes 11 of an inner side of the lens 1, then the photochromic anti-fog composite lens 2 does not have the holes. An adjustment device 12 protrudes from the inner side of the lens 1 and located beside each of the two installation holes 11 so as to adjust the angle of the photochromic anti-fog lens.

It is noted that the term “inner side” represents the side located close to the user's eyes, on the contrary, the term “outer side” represents the side located remote from the user's eyes.

While we have shown and described the embodiment in accordance with the present invention, it should be clear to those skilled in the art that further embodiments may be made without departing from the scope of the present invention.

What is claimed is:

1. A photochromic anti-fog lens comprising:
a lens and a photochromic anti-fog composite lens, the photochromic anti-fog composite lens includes a first transparent plastic plate and a second transparent plastic plate which is glued to an inner side of the first transparent plastic plate by glue, the glue having photochromic material, the second transparent plastic plate having an anti-fog film attached to an inner side thereof, the lens being formed in a molding set and securely connected with the first transparent plastic plate located in a cavity of the molding set located beside the inner side of the first transparent plastic plate.

2. The photochromic anti-fog lens as claimed in claim 1, wherein the lens, the first transparent plastic plate and the second transparent plastic plate each are made of the Polycarbonate.

3. The photochromic anti-fog lens as claimed in claim 1, wherein the first transparent plastic plate and the second transparent plastic plate each have a thickness of 0.1 mm to 0.5 mm.

4. The photochromic anti-fog lens as claimed in claim 1, wherein the first transparent plastic plate and/or the second transparent plastic plate includes photochromic material therein.

5. The photochromic anti-fog lens as claimed in claim 1, wherein the lens is adapted to be used as helmet goggles or ski goggles, the lens includes an installation hole defined in each of two ends thereof, the photochromic anti-fog composite lens is overlapped to an inner side of the lens, the photochromic anti-fog composite lens includes two holes which are located corresponding to the two installation holes.

6. The photochromic anti-fog lens as claimed in claim 1, wherein the lens is adapted to be used as helmet goggles or ski goggles, the lens includes an installation hole defined in each of two ends thereof, the photochromic anti-fog composite lens is overlapped to an area between the two installation holes of an inner side of the lens.

7. The photochromic anti-fog lens as claimed in claim 5, wherein an adjustment device protrudes from the inner side of the lens and located beside each of the two installation holes.

8. The photochromic anti-fog lens as claimed in claim 6, wherein an adjustment device protrudes from the inner side of the lens and located beside each of the two installation holes.

9. A method for making a photochromic anti-fog lens, comprising:
a step 1: mixing photochromic material in glue;
a step 2: spreading the glue onto an inner side of a first transparent plastic plate and/or an outer side of the second transparent plastic plate, attaching the second transparent plastic plate to the inner side of the first transparent plastic plate, attaching an anti-fog film on an inner side of the second transparent plastic plate to form a photochromic anti-log composite lens;
a step 3: putting the photochromic anti-fog composite lens in a cavity of a molding set, the first transparent plastic plate located at an inner side of a lens to be formed in the molding set, and
a step 4: injecting a melted lens-forming material into the cavity of the molding set, the melted lens-forming material connected with the first transparent plastic plate to form a photochromic anti-fog lens that includes the lens connected to the photochromic anti-fog composite lens.

10. The method as claimed in claim 9, wherein the step 1 further including a process of mixing the photochromic material with melted lens-forming material of the first transparent plastic plate and/or melted lens-forming material of the second transparent plastic plate so as to form the first transparent plastic plate having the photochromic material and/or the second transparent plastic plate having the photochromic material.

11. The method as claimed in claim 9, wherein when the lens is a curved lens and the photochromic anti-fog composite lens is a flat lens, the photochromic anti-fog composite lens is processed to be curved before the photochromic anti-fog composite lens is put into the cavity of the molding set such that the photochromic anti-fog composite lens is connected to the lens.

12. The method as claimed in claim 9, wherein the melted lens-forming material is 260 to 280 degrees Celsius when the melted lens-forming material is injected into the cavity of the molding set.
